(19) United States
(12) Patent Application Publication
(10) Pub. No.: US 2017/0283756 A1
(43) Pub. Date: Oct. 5, 2017

(54) MULTI-CHAMBERS BIOREACTOR, METHODS AND USES

(71) Applicant: ASSOCIATION FOR THE ADVANCEMENT OF TISSUE ENGINEERING AND CELL BASED TECHNOLOGIES, Braga (PT)

(72) Inventors: Raphaël FAUSTINO CANADAS, Guimarães (PT); Joaquim Miguel ANTUNES DE OLIVEIRA, Braga (PT); Alexandra Margarida PINTO MARQUES, Porto (PT); Rui Luís GONÇALVES DOS REIS, Porto (PT)

(21) Appl. No.: 15/512,306

(22) PCT Filed: Sep. 18, 2015
(86) PCT No.: PCT/IB2015/057210

§ 371 Date: Mar. 17, 2017

(30) Foreign Application Priority Data
Sep. 18, 2014 (PT) 107893

(51) Int. Cl. C12M 3/00; C12M 1/12; A61L 27/60; A61L 27/38; A61L 27/50; C12M 1/00; C12M 1/42

(52) U.S. Cl. CPC C12M 21/08; C12M 23/34; C12M 25/14; C12M 29/04; C12M 23/38; C12M 35/02; A61L 27/3821; A61L 27/3817; A61L 27/50; A61L 27/60; A61L 27/3813; A61L 27/3808; A61L 2430/02; A61L 2430/34

(57) ABSTRACT

The present disclosure relates to a multi-chamber bioreactor, preferably in a polymeric material with a 3D structure, adapted for cell-mono and co-culture, with at least two entries and outputs of culture medium adaptable to be used as a static culture system and to incorporate a dynamic platform creating a bioreactor. The disclosure also relates to a technique based on a bioreactor device that allows the creation of two or more different tissues integrated with the natural phenotype, using an integrated and continuous 3D support structure.

### TECHNICAL FIELD

The present disclosure relates to a multi-chamber bioreactor, preferably in a polymeric material with a 3D structure, adapted for cell-mono and co-culture, with at least two entries and outputs of culture medium adaptable to be used as a static culture system and to incorporate a dynamic platform creating a bioreactor.

The disclosure subject matter also relates to a technique based on a bioreactor device that allows the creation of two or more different tissues integrated with the natural phenotype, using an integrated and continuous 3D support structure.

### BACKGROUND ART

There are several different systems for 2D and 3D cell culturing namely, the static (e.g. tissue culture polysterene) and dynamic systems (e.g., bioreactors).

In the case of static culture systems, 2D, or flat culture plates, well culture plates, Petri dishes and T-flasks are the most common used technologies. There is also the possibility to combine both 2D systems, using a well culture plate with trans-well inserted to create a flat membrane over (and in the middle) of the well, allowing to have two different surfaces, interconnected, in the same culture well.

Recently new culture well plates are being created to be 3D. A mesh with a 3D architecture is designed in the bottom part of the well, allowing the cells to be cultured in a more biomimetic arrange, comparing to human body environment. A disposable chamber adapted to accommodate 3D structures was also created recently.

In tissue engineering and regenerative medicine there is a lack of physical structures to support multilayered and complex tissues. (Keeney and Pandit, 2009) The state of the art is characterized by several bilayered structures with sharp interfaces. (Nukavarapu and Dorcemus, 2012) It is extremely challenging to make a continuously gradient structure that allow smooth interface formation of two tissues with different interfaces, and one of the biggest problems is not related with the structure itself, but greatly dependent on the cell culturing conditions in a 3D environment. In this sense, there is a need for devices or bioreactors, enabling to induce the homogenization of the cells inside of the 3D structure or scaffolds, and at the same time, allowing having the optimal conditions for the production of multilayered tissue mimicking the native ones.

These facts are disclosed in order to illustrate the technical problem addressed by the present disclosure.

### GENERAL DESCRIPTION

In order to address the above mentioned technical problems and despites deficiencies related to the above solutions it is necessary to develop a reactor and a method to obtain synthetic tissues grafts with cells multilayers fused that mimic the native ones. Also has a further advantage that avoids the use of a binder between the synthetic tissues layers and improves the cell proliferation that can be evaluated by metabolic activity measurements.

Other problem is developed devices or bioreactors, enabling to induce the homogenization of the cells inside of the 3D structure or scaffolds, and at the same time, allowing having the optimal conditions for the production of multilayered tissue mimicking the native ones. To achieve this goal physical stimulus and appropriate nutrition/chemical cues (e.g., supply of culture medium with or without bioactive agents) shall be optimized for each region of the multilayered tissue.

One of the aspects of the present subject matter is a multi-chamber bioreactor with or without 3D structure, adapted for cell mono- and co-culture, with two entries and outputs of culture medium, adaptable to be used as a static culture system and also to incorporate a dynamic platform creating a bioreactor.

The present subject matter disclosed a multi-chamber bioreactor made in a polymeric material, namely plastic, with or without 3D structure, adapted for cell mono- and co-culture, with at least two entries and outputs of culture medium, adaptable to be used as a static culture system and to incorporate a dynamic platform creating a rotational bioreactor.

An aspect of the present disclosure relates to a polymeric multi-chamber bioreactor, preferably transparent, comprising
at least a first and a second fluid-tight chamber for receiving respectively a first fluid culture medium and a second fluid culture medium comprising respectively a first and a second cell culture, and an opening between the first and second chamber for interchanges between the chambers,
each chamber comprising at least an inlet and an outlet of fluid, preferably a fluid culture medium
the first and second chamber comprising respectively a first and a second cell culture scaffold,
wherein the first and second scaffold are in contact,
wherein the chamber and the scaffolds are arranged such that when the bioreactor is in use, an interchange of cells of the first, or of the second, or of both the first and the second cell culture occurs between the chambers through one or both scaffolds.

In another embodiment of multi-chamber bioreactor disclosed can comprise 2, 3, 4, 5 . . . 10, 20 . . . n chambers.

In another embodiment of multi-chamber bioreactor, the first and second scaffolds may be each one of the two layers of a bilayer scaffold.

In another embodiment of multi-chamber bioreactor, the pore size of the scaffolds is such to allow the interchange between the chambers of cells of the first cell culture, or of the second cell culture or of both the first and second cell culture.

In another embodiment of multi-chamber bioreactor, the pore size of the scaffolds is such to allow the interchange between the chambers of cells of both first or second cell culture. The size of a 3D cell culture—scaffolds—can be influenced by the ability to diffuse fresh culture medium, because the cells inner the 3D structure die if the fresh medium can't diffuse inside the structure, replacing the metabolic waste of the cells, which have an acidic nature.

Furthermore, the range of pore size is also dependent on the ability of the medium to enter and exit the interior of the 3D structure. Introducing the ability to have a stirring movement, the diffusion potential will increase, allowing increasing also the size of the 3D culture (resulting in the ability to produce larger tissues in vitro) and pore size range, allowing having a structure with smaller pore size and larger porosity and surface area.

In another embodiment of multi-chamber bioreactor, the pore size of the scaffold can be between 5 μm-2 mm; preferably between 10 μm-500 μm, more preferably 10 μm-49 μm. The pore size of the scaffold can be achieved by X-ray microtomography (micro CT), which perform an acquisition of the structure by X-ray in 180° or 360°, layer by layer and also reconstructing the structure three dimensionally, and have the ability to characterize several parameters as surface area, pore size, porosity, trabecular size and interconnectivity using several software analysis.

In another embodiment of multi-chamber bioreactor the porosity of the scaffolds can be between 10-98%, preferably between 70-90% which can be characterized also by micro CT.

In another embodiment of multi-chamber bioreactor, one or both scaffolds are of a polymeric, ceramic, hybrid or composite material, or combinations thereof; preferably in a transparent polymer.

In another embodiment of multi-chamber bioreactor, wherein one or both scaffolds are biodegradable, or chemically degradable, or photodegradable, or combinations thereof

In another embodiment of multi-chamber bioreactor, wherein one or both scaffolds may be degradable such that, when the bioreactor is in use, the interchange of cells occurs gradually.

In another embodiment of multi-chamber bioreactor the cultures are mono-cell cultures or cells co-cultures.

In another embodiment of multi-chamber bioreactor the first and second cultures are mono-cell cultures and/or cell co-cultures.

In another embodiment of the multi-chamber bioreactor one or more of the chambers can comprise a detachable cap.

In another embodiment of the multi-chamber bioreactor wherein the chamber cap can be suitable for compressing the respective cell culture and scaffold.

In another embodiment of the multi-chamber bioreactor wherein either the first or the second culture medium can be air.

In another embodiment of the multi-chamber bioreactor can further comprise pumping means wherein the chambers and the pumping means are arranged such that, when the bioreactor is in use, the flow of the fluids between each chamber inlet and the respective chamber outlet is laminar.

In another embodiment the multi-chamber bioreactor may further comprise a conductive metal, as a coating or filling the cell scaffold, in contact (direct or indirect) with the cell culture able to induce an electric pulsatile stimulus over the cell culture. This pulsatile pulse can be used as a stimulus to certain cell cultures but also to promote transfection of the cells by microporation.

In another embodiment the multi-chamber bioreactor may further comprise glycose, urea, pH, temperature, O2 pressure and CO2 pressure sensor, or combinations thereof.

In another embodiment the multi-chamber bioreactor may further comprise in the bottom part of said bioreactor a magnet. The magnet may be used to attract magnetics particles into the culture for transfection.

In another embodiment of the disclosed subject matter, the diffusion of culture medium of the multi-chamber bioreactor may be improved submitting said bioreactor to stirring movement (rotational movement). Namely the rotation can be promoted by magnetic stirring, having magnets performing the attraction between the bottom part of the bioreactor and the stirring position in a plate.

Another aspect of the present subject matter relates to the use of the multi-chamber bioreactor for obtain synthetic tissue grafts and/or in vitro tissue models for drug screening.

Another aspect of the present subject matter relates to a synthetic tissue graft obtainable by the use of the multi-chamber bioreactor comprising a multilayered tissue containing at least two different cell layers fused.

In another embodiment the synthetic tissue graft obtainable can be for: osteochondral interface tissue; a skin interface tissue; an intestine epithelial barrier; a blood-brain barrier; a lung epithelial barrier, among others.

The synthetic tissue graft of the present disclosure for use in human or veterinary medicine.

Another aspect of the present subject matter relates to the use of the synthetic tissue grafts disclosed for use in regenerative medicine and tissue engineering, in particular in the treatment of diseases that involve the regeneration, replacement or treatment of biologic tissues.

In another embodiment the multi-compartmentalized chambers allow flowing at least two different culture media. The multi-chambers are adapted for 3D structures that can support the growth of at least one or two different cell lineages (or stem cells fate).

In an embodiment, the dual culture chambers can have both, a porous central layer—a 2D or a 3D structure. This kind of chamber is used to culture cells with different conditions in each chamber. The design of the multi culture chambers allows avoiding the mixture of the culture media, having connection between the two compartments between the porosity of the central 3D porous structure. This 3D porous structure is designed to be easily discarded from the chamber, allowing to be analyzed independently.

Throughout the description and claims the word “comprise” and variations of the word, are not intended to exclude other technical features, additives, components, or steps. Additional objects, advantages and features of the present subject-matter will become apparent to those skilled in the art upon examination of the description or may be learned by practice of the invention. The following examples and drawings are provided by way of illustration, and they are not intended to be limiting of the present subject-matter. Furthermore, the present subject-matter covers all possible combinations of particular and preferred embodiments described herein.

### BRIEF DESCRIPTION OF THE DRAWINGS

The following figures provide preferred embodiments for illustrating the description and should not be seen as limiting the scope of present subject-matter.

FIG. 1. Perspective view of the dual chamber (with open view for the inside), wherein a) is top cap of each dual chamber, b) represents the central part of the chamber where is comprised the bi-compartmentalization with the central hole for structure insertion and c) represents the bottom part adapted for insertion of a magnetic bar to fix the dual chamber to the stirred plate by magnetic attraction.

FIG. 2. Cross section of the dual chamber.

FIG. 3. Represents examples of multilayered tissues and barriers in human body that could be mitigated by the present subject-matter; wherein: I—is skin layers; II—is osteochondral interface; III—is blood-brain barrier; IV—is Intestine epithelial barrier; V—is Lung epithelial barrier.

FIG. 4. Represents the metabolic activity obtained using the multi-chamber bioreactor of the present disclosure and a static culture condition.

FIG. 5. Cross section of the dual chamber, with a detail of the screw adapted to fit top hole of the dual chamber bioreactor, motor, and bottom view detail.

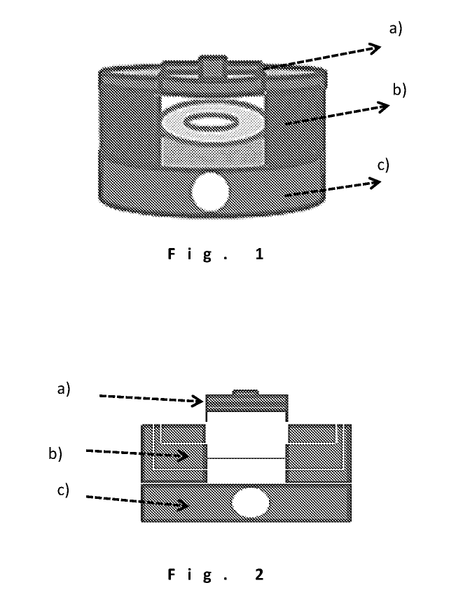

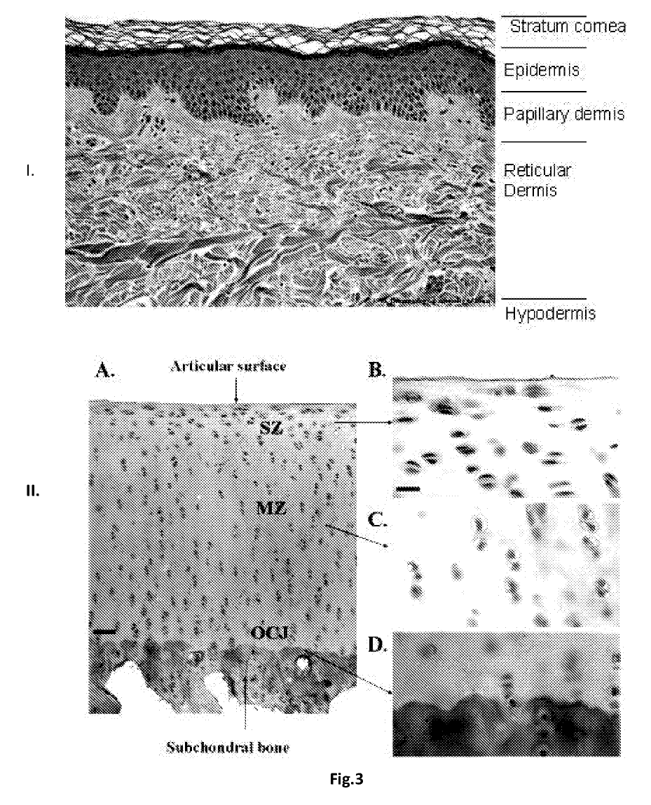

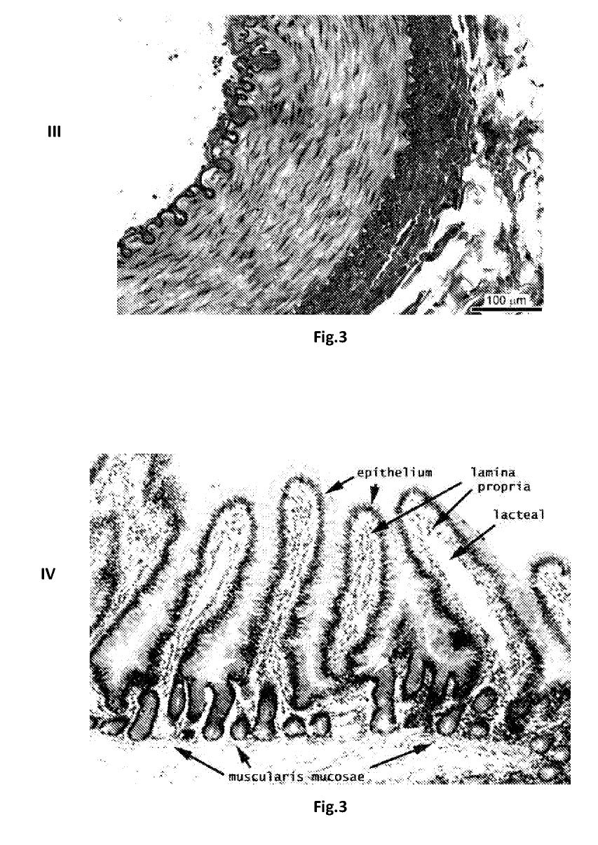

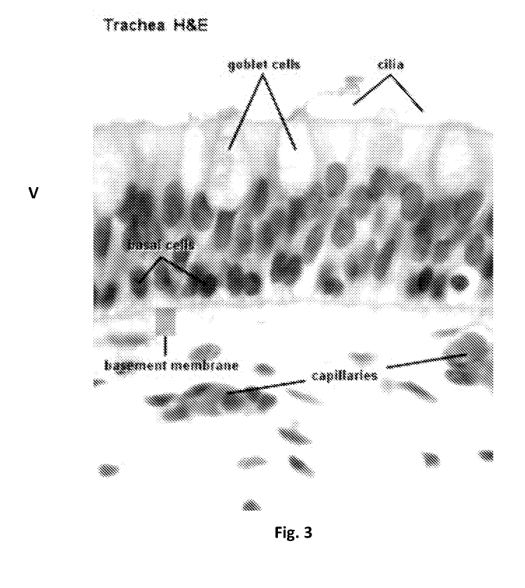

In another embodiment, in the bottom part of the dual culture chamber may be attached a magnetic bar to be attracted by the rotating position in a stirrer plate. This way the culture chamber can be used in both, static and dynamic conditions (in the last case, when attached to the stirrer plate). Each chamber may have detachable caps for the top and the bottom compartments. The top cap may present the possibly to adapt a compression drive, allowing to test a compressive stimulus over the cell culture.

In a another embodiment the plurality of chambers of the bioreactor may have dimensions to adapt to commercial 6-well tissue culture plates (38.4 mm diameter, 17.5 mm height). This way, and being the top and bottom of the chambers detachable, the culture can be observed by microscopy. Microsensors can be added to the system to monitor several biochemical parameters, as oxygen tension, pH, temperature, or glucose and urea concentration and physical parameters like pressure.

Different tissues such as bone and cartilage (osteochondral) and dermis and epidermis (skin), or barriers as, blood-brain, intestinal epithelium and lung epithelium, are examples of interfaces that should be integrated, present two or more different phenotypes in each side and are extremely difficult to be reproduced in the laboratory bench without and adapted system.

There is a need for devices or bioreactors, enabling to induce the homogenization of the cells inside of the 3D structure or scaffolds, and at the same time, allowing having the optimal conditions for the production of multilayered tissue mimicking the native ones. To achieve this goal physical stimulus and appropriate nutrition/chemical cues (e.g., supply of culture medium with or without bioactive agents) shall be optimized for each region of the multilayered tissue.

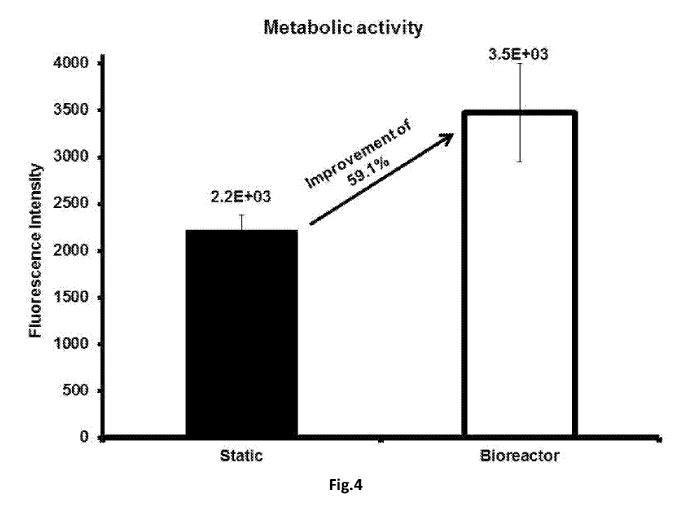

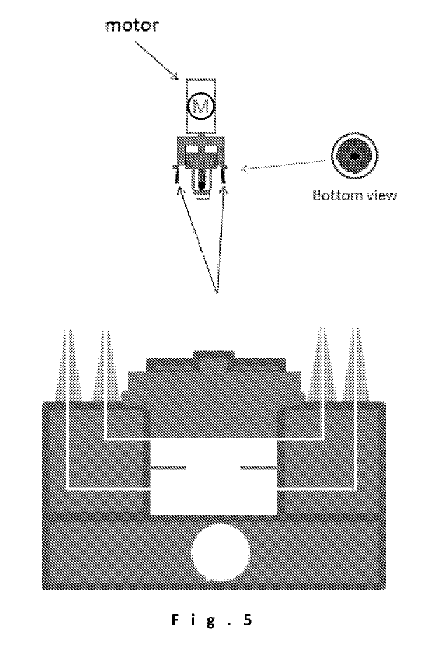

The metabolic activity is proportional to the cell number. In FIG. 4 the cells number at the seeding was the same in both 3D polymeric structures in static and dynamic (bioreactor) culture conditions. The structures were seeded with mesenchymal stem cells and cultured under static culture medium vs perfused culture medium. The perfusion of culture medium was performed in the multi-chamber bioreactor of the present disclosure. Using alamarBlue assay® the metabolic activity was measured by fluorescence, since resazurin is a molecule weakly fluorescent that is reduced into a fluorescent molecule by the cellular oxidation-reduction chain. The rate of metabolic activity is proportional to the fluorescence and so to the cell number. Comparing both conditions, an improvement of 59.1% in metabolic activity was obtained using the multi-chamber bioreactor of the present disclosure when compared with the static culture condition obtained using the static system (FIG. 4).

The present disclosure concerns a technique, based on a bioreactor device, to enhance the creation of continuous smooth gradient interfaces, able to create two or more different tissues integrated, with the natural phenotype, using an integrated and continuous 3D support structure.

Other aspect of the present subject-matter disclosed a technique, based on a bioreactor device, to enhance the creation of continuous smooth gradient interfaces, able to create two or more different tissues integrated, with the natural phenotype, using an integrated and continuous 3D support structure.

### DESCRIPTION OF PREFERRED EMBODIMENTS

An embodiment of the present disclosure refers to a rotational dual chamber bioreactor that is composed by a set of dual culture chambers interconnected, a multi-position magnetic stirrer plate, and flow pump(s).

The dual culture chambers present two independent culture medium entries and outputs, which permit induction of independent and different shear flows, and a central separator with a hole for insertion of the scaffold, whereby there is integration of the two chambers. The dual chambers have a magnetic bar attached to the bottom part.

The bioreactor disclosed may further comprise a multiposition magnetic stirrer plate with several position, for example 12 positions adapted for two 6 well tissue culture plates, which control independent horizontal movement for each position and vertical movement for all the plate.

In another embodiment, the dual chamber bioreactor further comprises a stirrer plate, which can be rotated vertically until 180°. In addition, the stirrer plate have 12 positions where can be placed, by magnetic attraction, 12 dual chambers. Each one of the 12 positions can be independently controlled to rotate until 180° with 10 different speeds ranging from zero to 0.12 sec/degrees at no load.

In another embodiment the dual chamber bioreactor may incorporate 12 dual culture chambers. The dual culture chambers may have a central barrier with a hole to insert the bilayer scaffold with a porosity no more than 50 μm, preferably 10-49 μm, more preferably 20-30 μm. This kind of well serve to culture cells with different conditions in each chamber. The design of the dual culture chambers allows avoiding the mixture of the culture media. In the bottom part of the dual culture chamber is attached a magnetic bar to be attracted with the rotating position in the stirrer plate. Each chamber has detachable caps for the top and the bottom chambers. The top cap presents the possibly to compress the scaffold, allowing to test a compressive stimulus. The dual chambers may have dimensions to adapt to commercial 6-well tissue culture plates, namely 11 to 39 mm diameter; in particular 38.4 mm diameter, 15-20 mm height, in particular 17.5 mm. This way, and being the top and bottom of the chambers detachable, the culture can be observed by microscopy. Microsensors can be added to the system to monitor several biochemical parameters, as oxygen tension, pH, temperature, glucose and urea concentration and physical parameters like pressure. All of the pieces that compose the dual chambers can be autoclavable.

Stirrer Plate:

In another embodiment, a stirrer plate can be adapted dimensionally to two standard 6 well tissue culture plates namely with 12 multiposition. Preferably, in each of the 12 positions can be inserted one of the dual culture chambers by magnetic attraction. Preferably, in each position can rotate until 180° (horizontal), being the rotations per minute controlled independently. The rotation is promoted by magnetic stirring, having magnets performing the attraction between the bottom part of the well and the stirring position in the plate. Vertical 180° movement can also be applied for all the chambers together.

In another embodiment, the stirrer plate can be controlled by a keyboard linked to a LCD display. The system is coordinated by an arduino (Atmel®) synchronized with a servo control module. The stirrer plate can also incorporate a wi-fi system to control the stirring at distance, using computer software.

All of the multi-chambers bioreactors disclosed may be placed inside an incubator.

Osteochondral (OC) Tissue Development Using the Dual Chamber Rotational Bioreactor

To develop an OC analogue, which can be further use in as an in vitro 3D tissue model, undifferentiated adipose derived stem cells (ASCs) isolated from Fat Pad are cultured in a bilayered scaffold, aiming at in situ cell differentiation into chondrocyte and osteoblast-like cells. The bilayered scaffolds comprise the cartilage- and bone-like layers, which are composed of gellan gum (GG) and GG with dispersed hydroxyapatite (HAp) particles, respectively.

In vitro mature and homogeneous OC tissue formation is achieved by culturing ASCs within the GG-HAp/GG bilayered scaffold by means of using the dual chamber rotational bioreactor. An optimized chemical mediation is provided at each compartment of the dual chamber, i.e. in one compartment is provided the osteogenic medium and in the second compartment is provided the chondrogenic medium. Chondrogenic differentiation culturing cocktail is performed based on well-established protocols.

Although osteogenic differentiation of ASCs can require additional Growth Factors, due to the presence of hydroxyapatite in the bone-like part of the scaffold, the conditions to maintain both cell types in co-culture needed to be optimized. The optimization, taking advantage of the dynamic culture system, accounts for the presence and the influence of osteogenic/chondrogenic mediators such as dexamethasone, L-ascorbic acid-2-phosphate, β-glycerophosphate, BMPs, FGF, platelet-derived GF and TGF-β.

The mixture of the different culture medium is prevented due to the independent flow through the two compartments of the chamber. This maximizes the differentiation potential of the ASCs towards each lineage in the respective scaffold layers. The system characterized by the dual chamber rotational bioreactor and the produced living tissue aims to be used as a 3D in vitro OC model. These 3D tissue models make possible the continuous analysis of the growth factors production and allow culture conditions optimization, thus holding a great promise for application in tissue engineering and regenerative medicine, and screening of bioactive molecules or drugs.

Development of an Osteochondral Human Tissue Interface

In an embodiment, o develop the osteochondral analogue (bone and cartilage interface), undifferentiated human adipose derived stem cells (hASCs) will be cultured in a bilayered system, aiming at in situ cell differentiation into chondrocyte and osteoblast-like cells. These hASCs can be derived from different tissues as the abdominal fat or from fat pad (also known as Hoffa's body) behind the knee. The osteogenic differentiation can be achieved by a specific chemical composition of the culture medium or be the physical stimulus from the structure in the bony part induced by the hydroxyapatite or nano-calcium phosphate particles dispersed within a silk-based structure (e.g.). An optimized chemical mediation that will be responsible for the chondrogenic differentiation of the hASCS encapsulated in methacrylated gellan gum (GG) incorporated in a silk backbone structure.

The goal is to integrate a bony part with good vascularization and enervation with an avascular cartilage layer. To promote this vascularization human adipose microvascular endothelial cells (hAMECs) can be co-cultured in the bone-like layer.

Also primary cells as osteoblasts and chondrocytes isolated from humans, for example with osteoarthritic phenotype, can be used to culture and create an in vitro disease model. This way would be possible to study the disease phenotype in a more realistic 3D structure.

To make possible the existence of the optimal environmental conditions to promote the production of these two layers continuously integrated, an adapted bioreactor is needed. The bioreactor will allow the culture of both layers integrated in two different chemical mediums. Moreover, the cartilage-like layer will be cultured in a lower flow perfusion medium and hypoxic conditions. The culture dual-chamber will be under 180° stirring to improve culture medium diffusion to inside and outside the 3D structure. The entire system will turn in 180° up and down to promote cell homogenization within the 3D structure, avoiding cell sedimentation, contributing to tissue maturation.

Development of Skin Tissue with Dermis and Epidermis

In addition a skin model can be developed; an epidermal analogue will be created by culturing hASCS-derived epidermals in a GG-keratin membrane, while the dermal analogue aiming at vascularized neodermis formation will be achieved by co-culturing hASCs and hAMECs in a GG-Hyaluronic acid matrix. The goal is to achieve a continuous epidermis supported by hASCs, and dermis vascularization promoted by hAMECs.

In order to create an environment that more realistically represent an in vivo situation, the dynamic bioreactor will be used once again using the flow pump to induce the culture medium flow linked to the rotational dual-chamber. The use of the bioreactor may improve tissue interface standardization, with a continuous and controlled medium composition. To improve skin tissue maturation an air-liquid interface can be created using the rotational dual-chamber bioreactor, allowing to flow an optimized culture medium in dermis layer and having an air phase in epidermis layer, supported by the dual-chamber.

This bioreactor system will also make possible to continuously analyse growth factors production and allow culture conditions optimization.

The proposed engineered tissues can be used as in vitro models and will be validated after performance comparison with parallel in vivo models. Ultimately these models are of great interest to predict in vitro TE constructs outcome, avoiding superfluous in vivo trials and minimizing their variability by providing controlled testing conditions. Furthermore these engineered tissues will be the first step to produce human tissues in lab with the final goal of transplantation.

Validation of the In vitro Models

In order to validate the developed models, in vivo tests will be performed in rat osteochondral and full-thickness excisional skin models. Similar defects will be produced in the in vitro (our developed models) and in vivo models and clinically available TE products will be tested. OrCel™ Bilayered Cellular Matrix, a bilayered cellular matrix in which normal human allogeneic skin cells (epidermal keratinocytes and dermal fibroblasts) are cultured in two separate layers will be tested regarding skin regeneration. Chondro-Gide®, consisting in an acellular membrane with a multilayer structure formed by collagen types I and III, with one compact and one porous side will be tested in the osteochondral model. The performance of the products in terms of tissue regeneration will be compared between in vitro and in vivo models based on type/composition and organization of deposited ECM, cellular organization within the different layers, vascularisation of the bone and dermal/hypodermal layers. These results will allow us to ensure that the proposed models could be used to test in vitro skin and osteochondral TE constructs, thus minimizing animal experimentation.

The disclosure is of course not in any way restricted to the embodiments described and a person with ordinary skill in the art will foresee many possibilities to modifications thereof.

The above described embodiments are obviously combinable.

The following claims further set out particular embodiments of the disclosure.

1. A polymeric multi-chamber bioreactor comprising
at least a first and a second fluid-tight chamber for receiving respectively a first fluid culture medium and a second fluid culture medium comprising respectively a first and a second cell culture, and an opening between the first and second chamber for interchanges between the chambers,
each chamber comprising at least an inlet and an outlet of fluid,
the first and second chamber comprising respectively a first and a second cell culture scaffold,
wherein the first and second scaffold are in contact,
wherein the chamber and the scaffolds are arranged such that when the bioreactor is in use, an interchange of cells of the first, or of the second, or of both the first and the second cell culture occurs between the chambers through one or both scaffolds;
wherein the pore size of the scaffolds is such to allow the interchange between the chambers of cells of the first cell culture, or of the second cell culture or of both the first and second cell culture.

2. The multi-chamber bioreactor according to claim 1 further comprising pumping means wherein the chambers and the pumping means are arranged such that, when the bioreactor is in use, the flow of the fluids between each chamber inlet and the respective chamber outlet is laminar.

3. The multi-chamber bioreactor according to claim 1 wherein the first and second scaffolds are each one of the two layers of a bilayer scaffold.

4. The multi-chamber bioreactor according to claim 1 wherein the pore size of the first scaffold, or of the second scaffold, or of both the scaffolds is between 5 μm-2 mm; preferably between 10 μm-500 μm, more preferably 10 μm-49 μm.

5. The multi-chamber bioreactor according to claim 1 wherein the porosity of the scaffolds is between 70-90%.

6. The multi-chamber bioreactor according to claim 1 wherein one or both scaffolds are of a polymeric, ceramic, hybrid, composite material or combinations thereof.

7. The multi-chamber bioreactor according to claim 1 wherein one or both scaffolds are biodegradable, or chemically degradable, or photodegradable, or combinations thereof.

8. The multi-chamber bioreactor according to claim 1 wherein one or both scaffolds are degradable such that, when the bioreactor is in use, the interchange of cells occurs gradually.

9. The multi-chamber bioreactor according to claim 1 wherein the first and second cultures are mono-cell cultures.

10. The multi-chamber bioreactor according to claim 1 wherein the first and second cultures are cell co-cultures.

11. The multi-chamber bioreactor according to claim 1 wherein one or more of the chambers comprises a detachable cap.

12. The multi-chamber bioreactor according to claim 1 wherein the chamber cap is suitable for compressing the respective cell culture and scaffold.

13. The multi-chamber bioreactor according to claim 1 wherein either the first or the second culture medium is air.

14. The multi-chamber bioreactor according to claim 1 further comprising a conductive metal in contact with the cell culture able to induce an electric pulsatile stimulus over the cell culture.

15. The multi-chamber bioreactor according to claim 1 further comprising glycose, urea, pH, temperature, O2 pressure CO2 pressure sensors, or combinations thereof.

16. The multi-chamber bioreactor according to claim 1 further comprising in the bottom part of said bioreactor a magnet.

17. (canceled)

18. A synthetic tissue graft formed by the multi-chamber bioreactor of claim 1 which comprises a multilayered tissue containing at least two different cell layers fused.

19. The synthetic tissue graft according to claim 18 wherein the synthetic graft is synthetic osteochondral interface tissue, or synthetic skin interface tissue, or synthetic intestine epithelial barrier, or synthetic blood-brain barrier, or synthetic lung epithelial barrier.

20. The synthetic tissue graft according to claim 19 for use in human or veterinary medicine.

21. (canceled)

22. (canceled)
